(19) United States
(12) Patent Application Publication
(10) Pub. No.: US 2003/0213715 A1
(43) Pub. Date: Nov. 20, 2003

(54) Biological material package with viewable temperature indicator

(76) Inventors: Gary W. Klepac, Hutto, TX (US); Charles L. Simpson, Austin, TX (US)

Correspondence Address: WILLIAMS, MORGAN & AMERSON, P.C., 10333 RICHMOND, SUITE 1100, HOUSTON, TX, 77042, US

(21) Appl. No.: 10/145,465

(22) Filed: May 14, 2002

(51) Int. Cl. A61L 15/00

(52) U.S. Cl. 206/438; 206/459.5; 206/524.8; 206/524.5

(57) ABSTRACT

The present invention provides a biological material package comprised of an enclosed compartment and a temperature sensor capable of sensing temperature extremes within the enclosed storage compartment. The temperature sensor is capable of providing a visual indication of a temperature extreme occurring within the enclosed compartment. The indication of the temperature extreme is visible from outside of the enclosed compartment, without opening the enclosed compartment.

### BACKGROUND

The present invention relates to biological material packages and more particularly to biological material packages having an improved temperature extreme indicator arrangement.

Implantable devices containing biological materials must be protected from potentially harmful temperature extremes during long periods of shipping and/or storage. In order to avoid potentially damaging effects arising from temperature extremes, various shipping and storage devices have been devised to protect biological materials. For example, if a bioprosthetic heart valve is exposed to freezing temperatures, there is a potential for exposure to undetectable ice crystals, which may form within the valve tissue causing damage.

To address this problem, most commercially available packages for implantable biological material devices utilize temperature indicators that, when exposed to a given temperature extreme, provide a visible signal such as a color change or ink dot to indicate that the package has been exposed to an extreme temperature. A predetermined temperature extreme triggers the signal. The temperature extreme may be either a predetermined high or low temperature or both high and low temperatures. Such indicators are usually “one-way” indicators in which the visible signal will remain in the changed condition even if the temperature returns to the acceptable range. By using one-way indicators, an inspector can readily determine if the package has been exposed to an extreme temperature at any time during the shipping/storage period.

A survey of commercially available packages for biological materials reveals that most such devices use indicators that detect a low temperature such as zero degrees centigrade. However, these indicators are located in a variety of different locations within the packaging systems, each of which has undesirable limitations.

One device for storing biological heart valves employs a freeze indicator on the outside of a polystyrene foam container that contains a jar for holding the valve. Another biological heart valve has a freeze indicator within an outer carton but outside of a two piece polystyrene foam container that encloses a valve jar. Still another device utilizes an oversized polystyrene foam container housing an inner carton. Where the temperature indicator is outside of the insulated container, the unprotected indicator could be prematurely tripped by temperatures occurring at the outside of the container, even though the biological material, within the container, is protected from the temperature extremes and thus never subjected to the undesirable environment. A signal tripped in this manner results in unwarranted rejection of undamaged biological material. In another commercially available container, a temperature indicator is located inside the insulated container but the insulated container must be opened to view the indicator, requiring time-consuming unwrapping and rewrapping of the valve and risking damage during such handling.

Therefore, there is a need for a package that allows protection of biological materials with a temperature sensor which is viewable and readable without opening the package and which indicates temperature extremes within the package compartment in which the biological material or a container for the biological material is stored.

### SUMMARY

The present invention provides a biological material package comprised of an enclosed compartment and a temperature sensor capable of sensing temperature extremes within the enclosed compartment. The temperature sensor is capable of providing a visual indication of a temperature extreme occurring within the enclosed compartment. The indication of the temperature extreme is visible from outside of the enclosed compartment, without opening the enclosed compartment.

An important advantage of the package is that a distributor in a warehouse, medical personnel in a hospital, or any other person desiring to verify the integrity of the device can, during shipment or storage, easily check the temperature sensor without compromising the integrity of the enclosed package. The placement of the temperature sensor minimizes the risk of prematurely tripping the temperature sensor. The package may also be designed to insulate the enclosed compartment to reduce the possibility of exposing the biological material itself to temperature extremes.

In another embodiment, the invention comprises a combination medical device and package comprising a closed compartment having a temperature sensor capable of sensing temperature extremes in the compartment. The sensor provides a signal visible from outside the compartment.

### BRIEF DESCRIPTION OF THE DRAWINGS

FIG. 1 is a cross-sectional side view illustrating an embodiment of a biological product package according to an embodiment of the invention.

FIG. 2 is a plan view illustrating an unfolded outer carton for use in connection with the package of FIG. 1.

FIG. 3 is a cross-sectional side view illustrating an embodiment of a biological material package according to another embodiment of the present invention.

FIG. 4 is a cross-sectional side view illustrating an embodiment of a biological material package according to another an embodiment of the present invention.

FIG. 5 is a cross-sectional side view illustrating an embodiment of a biological material package according to yet another embodiment of the present invention.

### DETAILED DESCRIPTION

In one embodiment, the present invention provides a biological material package comprising a multi-piece package forming an enclosed compartment. The package has a first piece and a second piece which may be separated from each other to open the enclosed compartment. The first piece and the second piece define a compartment for receiving the biological material, preferably in a sealed container or jar. One of the first or second pieces of the package comprises a view port that extends from an external surface of the respective first or second piece to the compartment, thereby providing a passageway between the external surface and the storage compartment containing the biological material. A temperature sensor is preferably provided in the passageway adjacent the storage compartment. In an alternate embodiment, the temperature sensor may be provided within the storage compartment. A window, which may be insulated, is preferably provided to allow the sensor to be viewed through the view port without opening the package. The window is provided on the external surface of the first or second piece comprising the view port. In another embodiment, the window is provided in the passageway. In an alternative embodiment, the window may be provided in the compartment.

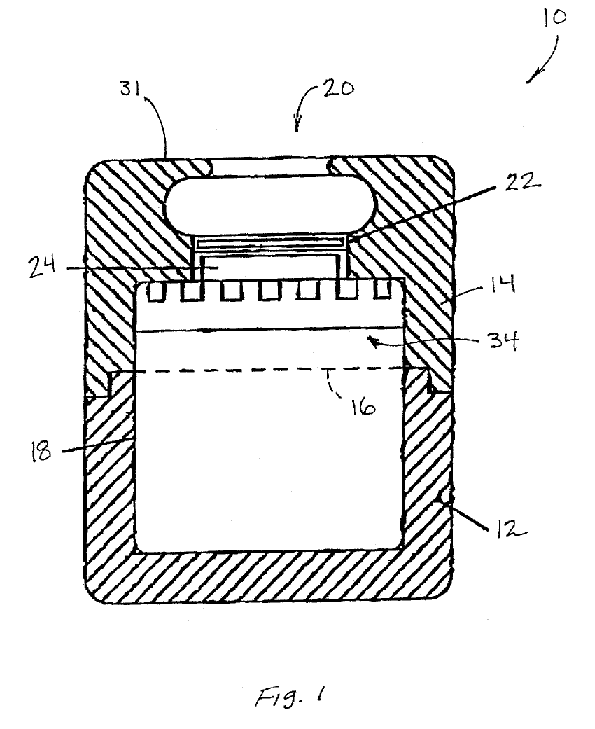

FIG. 1 depicts an embodiment in which the biological material package is generally designated 10. The package comprises a closed container having a first piece 12 and a separable second piece 14 coupled to the first piece. In one embodiment, first and second pieces 12 and 14 have a nested engagement at an interface 16. However, it is understood that biological material packages may comprise fewer or more pieces than the two-piece construction depicted in FIG. 1 without departing from the scope of the invention. Returning to FIG. 1, the first piece 12 and the second piece 14 when coupled together define a storage compartment 18. The second piece 14 includes a view port 20 extending through an exterior wall 31 of piece 12 to provide a view into storage compartment 18. A biological material container 34 is shown in place in storage compartment 18.

Package 10 further comprises a temperature sensor 24. The temperature sensor may be selected from a variety of commercially available temperature sensors known in the art. The temperature sensor 24 is preferably located adjacent to container 34 residing in compartment 18. An insulated window 22 may be provided in the view port 20 so as to better insulate the sensor 24 from temperature extremes occurring outside package 10. The insulating window also allows the temperature sensor 24 to be viewed without opening the package 10, i.e., without separating or otherwise uncoupling the second piece 14 from the first piece 12.

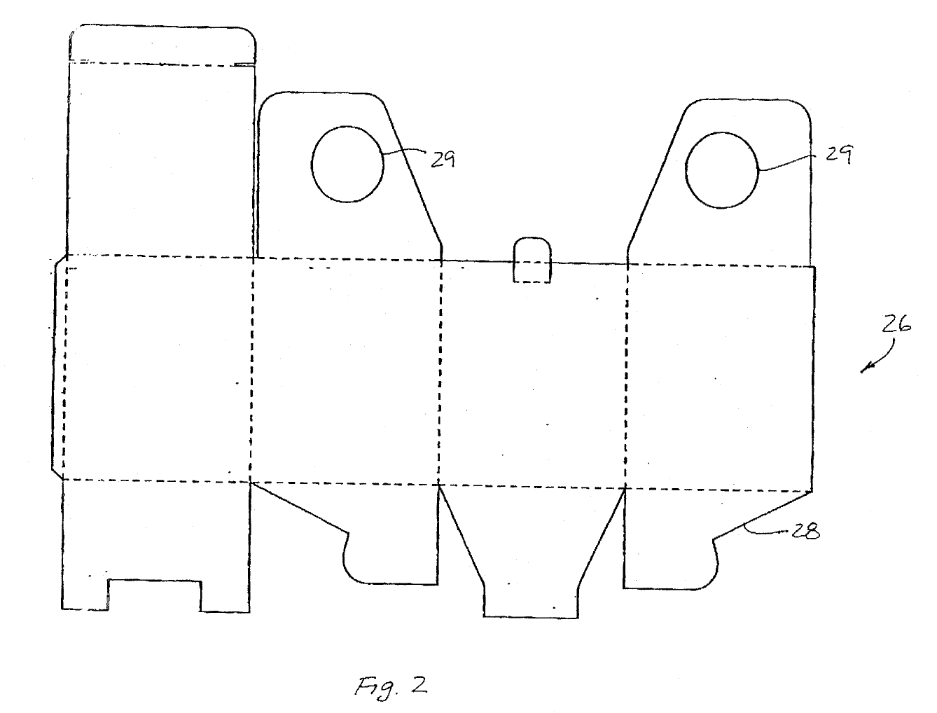

Aspects of additional embodiments of the present invention are depicted in FIGS. 2, 3, 4, and 5 in which reference numbers consistent with those used in FIG. 1 identify like components of the different embodiments. If desired, the view port 20 may be provided in the first portion 12. Usually it is desirable that the package have insulating properties to protect the storage compartment, and particularly the biological material therein, to the greatest extent possible. However, it is understood that a non-insulated package is within the contemplation of the present invention.

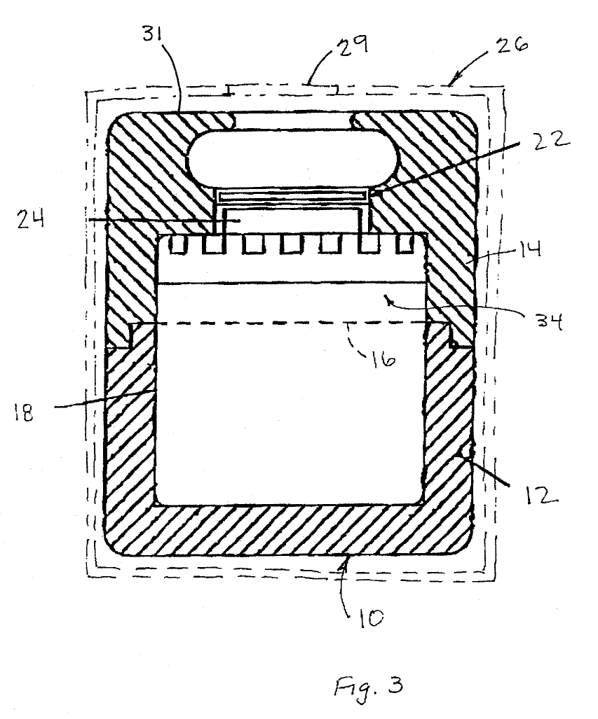

As depicted in FIGS. 2 and 3, a carton 26, such as a removable shipping carton, may be formed from a foldable blank 28 and used to house the package 10. Carton 26 comprises a window or aperture 29 that may be aligned with view port 20 and insulated window 22 to maintain the visibility of sensor 24.

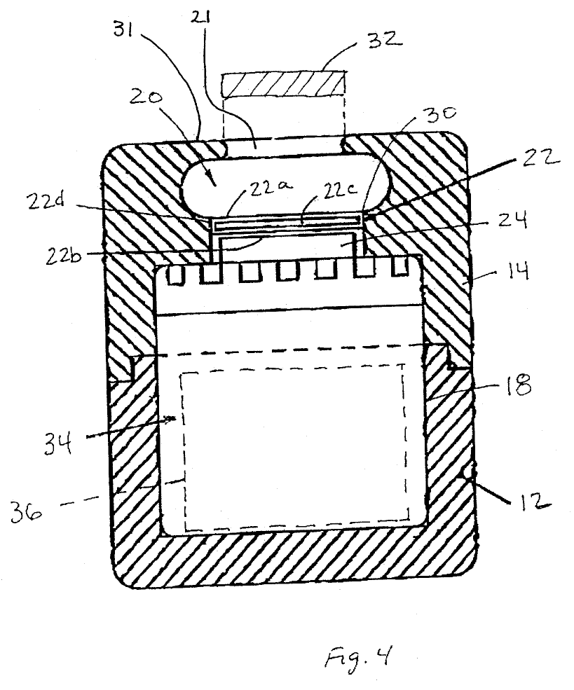

In another embodiment in accordance with the present invention, the package, as shown in FIG. 4, is fabricated of any suitable packaging material such as expanded polystyrene foam. The window 22 may include a first transparent member 22a and a second transparent member 22b separated by an insulating air gap 22c. The view port 20 includes an aperture 21 and a passageway 30 in second portion 14, and the window 22 includes a peripheral edge 22d engaged with the passageway 30. Also, a removable insulated foam or clear polymeric plug 32 may be provided for seating in aperture 21 to the view port 20, if desired.

The storage compartment 18 is provided for receiving the biological product container 34 which contains a temperature sensitive biological material, or a biologically derived material 36. The material 36 may comprise a heart valve, a graft, a patch or another implant containing biological materials. In a preferred embodiment, temperature sensor 24 is a TeleTemp ColdSNAP+Dual Temperature Recorder available from TeleTemp Corp., Fullerton, Calif.

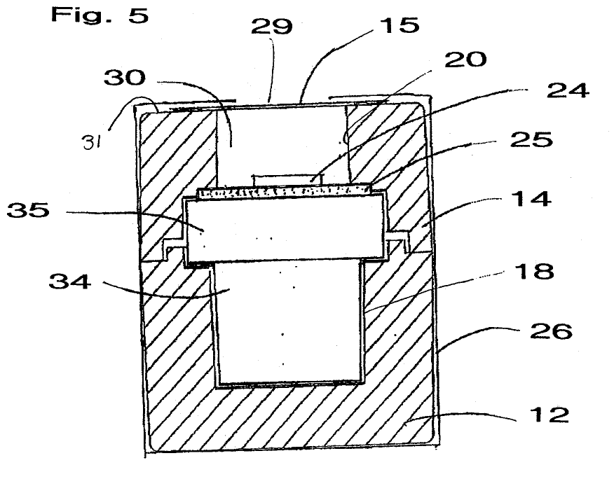

A further embodiment of the present invention, depicted in FIG. 5, provides an insulated biological material package including a first piece 12 and a second piece 14, defining a storage compartment 18. The insulated package houses a biological product container 34 having a screw-on cap 35. A temperature sensor 24 is positioned adjacent to the storage compartment. The temperature sensor is mounted on a substrate such as a foam card 25, which together with the walls of the storage compartment provide an enclosed storage compartment.

A first view port 20 in the second piece 14 of the insulating package exposes the sensor to viewing from outside of the package. An insulated window 15 is coupled to view port 20 at or adjacent to the exterior wall 31 of second piece 14, to help insulate the storage compartment and to permit viewing of the temperature sensor. Additionally, an enclosed air space between the insulated window and the temperature sensor/substrate combination provides an insulating enclosed air space 30. A carton 26 houses the insulating package, and a second view port 29 in the carton aligned with the first view port 20 allows the sensor to be viewed without opening the package while it is in the carton.

The materials suitable for construction of packages in accordance with the present invention should have structural strength and shock absorbing characteristics sufficient to protect the packaged biological material from mechanical damage during shipment and storage. It has been found that polystyrene foam having a thickness of at a least about 2 cm is suitable for such purposes. However, it is understood that other thicknesses and other materials are suitable for construction of packages in accordance with the present invention.

For construction of insulated packages in accordance with the present invention, the insulating materials should have insulating characteristics sufficient to protect the packaged biological material from temperature extremes likely to occur during shipment and storage. The polystyrene foam materials mentioned above are suitable for such purposes. However, it is understood that other thicknesses and other materials are suitable for construction of insulating packages in accordance with the present invention.

Biological material packages in accordance with the present invention may also include designs without view ports. For example, the packages may incorporate temperature sensors capable of sensing temperature extremes within the enclosed compartment, but instead of providing a direct visual indication of the temperature sensor through a view port, packages according to the present invention may provide a visual indication detectable from outside of the enclosed compartment in a different manner. For example, packages in accordance with the present invention may incorporate an electric wire or other component capable of transmitting a signal to activate a visual indication outside of the enclosed compartment upon the occurrence of a temperature extreme within the enclosed compartment.

In preferred embodiments, the present invention has the advantage that the packages can be shipped or stored with a viewable temperature sensor. This allows a distributor, hospital personnel, or anyone desiring to verify the integrity of the biological materials to check the temperature sensor without compromising the integrity of the closed, insulated package, which may also be sealed to prevent tampering. The packages also minimize the chances of prematurely tripping the temperature sensor. The packages may also be designed to insulate the enclosed compartment to reduce the possibility of exposing the biological material itself to temperature extremes.

Although illustrative embodiments have been shown and described, a wide range of modifications, changes and substitutions is contemplated in the foregoing disclosure. In some instances, certain features may be employed without a corresponding use of other features. Variations of the present invention will be apparent to those skilled in the art. Such variations are considered within the spirit and scope of the claimed invention.

What is claimed is:

1. A biological material package comprising:
(a) a package comprising an enclosed compartment; and
(b) a temperature sensor capable of
(1) sensing the temperature within the enclosed compartment and
(2) providing a visual indication of a temperature extreme, said indication being visible from outside the enclosed compartment.

2. The biological material package of claim 1 wherein the package comprises multiple pieces.

3. The biological material package of claim 2 wherein the visual indication of the temperature extreme is visible from outside the enclosed compartment through a view port.

4. The biological material package of claim 3 comprising a container containing biological material within the enclosed compartment.

5. A biological material package comprising:
(a) a plurality of pieces coupled together to form a package having an exterior wall and a storage compartment;
(b) a temperature sensor coupled to the package in a location selected from the group consisting of within the view port and within the storage compartment; and
(c) a view port through which the temperature sensor is visible, said view port extending from the exterior wall of the package to the storage compartment of the package.

6. The biological material package of claim 5 further comprising a first window member provided in a location selected from the group consisting of along the exterior wall of the package and within the view port between the temperature sensor and the exterior wall, the temperature sensor being visible through said first window member from the exterior wall.

7. The biological material package of claim 5 wherein the package comprises an insulating material.

8. The biological material package of claim 5, further comprising a first, transparent window member, a second transparent window member, and an air gap between the first window member and the second window member.

9. The biological material package of claim 6, wherein the view port comprises a passageway in the package, and the first window member is provided in the passageway.

10. The biological material package of claim 5 further comprising a removable insulating plug positioned in the view port adjacent said exterior wall.

11 The biological material package of claim 5 further comprising a container enclosed in the storage compartment.

12. The product package as defined in claim 11 wherein the container contains biological material.

13. The biological material package of claim 12 wherein the biological material is a heart valve.

14. The biological material package of claim 12 wherein the biological material is a graft.

15. The biological material package of claim 12 wherein the biological material is a patch.

16. The biological material package of claim 5 further comprising a removable carton enclosing the package.

17. The biological material package of claim 6 wherein the carton comprises an aperture, aligned with the first window member, through which the temperature sensor is visible.

18. The product package as defined in claim 7 wherein the insulating material comprises polystyrene foam.

19. An insulated biological product package comprising:
(a) an insulating package comprising an exterior wall and an enclosed storage compartment;
(b) a view port in the insulating package;
(c) a temperature sensor positioned adjacent to the enclosed storage compartment and visible through the view port;
(d) a window member coupled to the view port at said exterior wall; and
(e) an enclosed air space between the window member and the enclosed storage compartment.

20. The biological product package of claim 19 further comprising:
(a) a carton housing the insulating package; and
(b) an aperture in the carton aligned with the view port.
